(19) United States
(12) Patent Application Publication
(10) Pub. No.: US 2009/0137612 A1
(43) Pub. Date: May 28, 2009

(54) Treatment with combined use of oxypurines and ascorbate to prevent and mitigate iron-catalized oxidative damage in Alzheimer's disease and other neurodegenerative disorders

(76) Inventor: William Howard Waugh, Greenville, NC (US)

Correspondence Address: LYNN E BARBER, P O BOX 16528, FORT WORTH, TX, 76162, US

(21) Appl. No.: 11/986,922

(22) Filed: Nov. 27, 2007

(51) Int. Cl. A61K 31/522; A61K 31/34; A61P 25/28; A61P 25/00

(52) U.S. Cl. 514/263.31; 514/474

(57) ABSTRACT

A method is provided for the prevention and treatment of selective progressive degeneration within the central nervous system caused by hydroxyl-free or ferryl-free radicals formed by Fenton-type catalyzed reactions between diffusible hydrogen peroxide and localized bivalent iron. The invention embodies unique pharmacologic composition for antioxidant protection by oral supplementation with hypoxanthine conjointly with either sodium L-ascorbate or L-ascorbic acid. The hypoxanthine is provided for its sodium-dependent intestinal absorption and transport for the systemic production of higher antioxidant and iron-chelating uric acid levels. Ascorbate is provided as potent antioxidant to raise body ascorbic acid levels concurrently and to protect against possible deleterious effect from nucleobase or other molecular injury induced by oxidized uric acid as urate anion free radical caused in the antioxidant action of the uric acid. It is contemplated that such oral supplementation conjointly with hypoxanthine and L-ascorbate will support better health and will mitigate the progressive oxidative neuronal damage in Alzheimer's disease, amnestic mild cognitive impairment, Down syndrome, amyotrophic lateral sclerosis, and Parkinson's disease.

### TECHNICAL FIELD

This invention is in the field of orthomolecular medicine for the preservation of good health and the mitigation of degenerative disease by a novel means of antioxidant nutritional supplementation. The novel means is the combined use orally of hypoxanthine (6-oxypurine) as precursor to endogenous uric acid (2,6,8-oxypurine) and sodium L-ascorbate or L ascorbic acid (vitamin C) for the intestinal absorption of hypoxanthine combined with the absorption of ascorbic acid for the body formation of more uric acid and for more ascorbic acid as major extracellular fluid hydrophilic antioxidants in humans. This useful method is to prevent or mitigate bivalent iron-catalyzed oxidations by hydrogen peroxide formed endogenously that can produce highly-reactive hydroxy free radicals, which react very quickly to oxidize organic molecules in their vicinity in various diseases including Alzheimer's disease.

### BACKGROUND OF THE INVENTION

It is well known in the art that superoxide anion free radical and hydrogen peroxide (H₂O₂) in aerobic respiration may be toxic to cells and tissues. Superoxide free radicals are formed in the activity of electron transport chains in mitochondria and also in endoplasmic reticulum in cells by the “leaking” of electrons directly from reduced electron transfer carriers onto molecular oxygen (Halliwell B, 1989). Some oxidases also produce superoxide free radicals when reduced flavin in these enzymes is oxidized univalently by molecular ozygen (Halliwell B, 1989). Superoxide dismutases remove superoxide free radicals by converting them into hydrogen peroxide molecules which are not very reactive by themselves as oxidizing agents (Halliwell B, 1989). Hydrogen peroxide is also produced by red blood cells which form this peroxide from generated superoxide free radical upon release of oxygen from oxyhemoglobin (Waugh W H, 2003).

Hydrogen peroxide is known in the art to be toxic even in low micromolar concentration if it combines with bivalent (reduced) iron in the Fenton chemical reaction, to form the very highly-oxidizing hydroxyl free radical (Halliwell B, 1989). The initial product of bivalent iron and hydrogen peroxide is likely another highly-oxidizing species, an iron-oxygen complex call ferryl radical (Halliwell B, 1989; Imlay J A et al., 1988). Hydroxyl radicals react at great speed with almost every molecule found in living cells including DNA (causing strand breakage and chemical alterations in the purine and pyrimidine nucleobases), membrane lipids, and carbohydrates (Halliwell B, 1989). Hydrogen peroxide is diffusible and is capable of reacting with bivalent iron bound at or close to DNA to form hydroxyl radicals (Halliwell B, 1989). Cell chromatin with nucleoproteins is known to be rich in iron.

Areas of the human brain are rich in iron and the brain is poor in catalase, an enzyme which converts hydrogen peroxide to water and ground state oxygen (Halliwell B, 1989). However, there are high concentrations of ascorbic acid in the grey and white matter of the central nervous system and there is a greater cerebrospinal fluid ascorbate level than the ascorbate level in human plasma (Halliwell B, 1989). Ascorbic acid has long been know to be arguably the most important hydrophilic biological antioxidant (Hendler S S H, Rovik D, 2001). It can scavenge both reactive oxygen species and reactive nitrogen species and it acts to reduce the transition metals, iron and copper (Hendler S S H, Rovik D, 2001).

Alzheimer's disease (AD) has become one of the major health problems in the United States and the entire developed world. The enormous formal and informal costs of caring for patients with AD would be dramatically reduced if the clinical onset of AD were delayed. Because the presence of AD clinically doubles with every 5 years of age after 60, delaying onset by 5 years would reduce the prevalence of AD by half (Evans D A et al., 1989; Brockmeyer R et al., 1998). Prevention of mild cognitive impairment (MCI) preceding diagnosable AD and declines in AD dementia will probably be most effective when the intervention targets a process closely relevant to the disease pathogenesis (Espeland M A et al. 2006).

Alzheimer's disease is a brain disease, know for over 100 years, which causes progressive cognitive impairment and dementia. It is characterized in post mortem studies by the cardinal neuropathologic signs of intracellular and extracellular protein aggregates (tau-protein and amyloid beta-peptide abnormalities) in neurofibrillary tangles and neuritic amyloid plaques, respectively (Goedert M et al., 2006). Various treatments have been proposed in clinical trials, including antioxidants, even combined with ascorbic acid but not with uric acid or a nucleobase precursor of uric acid (Christen Y, 2000; Roberson E D et al., 2006).

An imbalance between the generation of free radicals and the accumulation of reactive oxygen species (ROS) has been suggested for many years in the pathogenesis of many neurodegenerative diseases including AD (Christen Y, 2000). Recently, strong evidence has been published that oxidative stress-mediated damage in cerebral tissue involving oxidation of nucleic acids, proteins, and lipids occurs in the early stages of AD, which precede the cardinal neuropathologic signs of AD (Nunomura A et al., 2006). Also, recent studies have shown that subjects with amnestic mild cognitive impairment have damage involving lipid peroxidation and DNA, RNA, and protein oxidative changes in multiple brain regions. These findings establish that free radical -mediated oxidative damage is involved as an early event in the neuron damage in AD (Markesbery W E et al., 2007).

In 1953, Goodman proposed the theory that iron accumulation was toxic and important in the pathogenesis of AD (Goodman L, 1953). He described numerous deposits of iron in many cerebral grey matter regions with iron deposits in the cytoplasm of neurofibrillary tangle-bearing neurons and with iron staining in amyloid plaques, oligodendroglia, and microglia. This iron burden theory is supported by recent workers including Connor J R et al. 1995, Smith M A et al. 1997, and Todovich B M et al. 2004. These workers concluded that iron accumulation and the cerebral iron-regulating system are dysfunctional in AD. Overaccumulation of iron was present in the hippocampus, cerebral cortex, and the basal nucleus of Meynert (Todovich B M et al., 2004). These affected brain areas are of special interest because they represent centers of memory and thought processes, all lost in the clinical picture of AD. Notably, iron has been reported elevated in the cerebrospinal fluid in AD compared to other control elderly patients (Hershey C O et al., 1983). Waugh recently proposed that cerebral iron homeostasis with iron accumulation is a likely primary seminal event in AD, by Fenton-type reaction of bivalent iron with hydrogen peroxide in forming the reactive hydroxyl-free radical or ferryl-free radical (Waugh W H, 2007).

Uric acid is a very strong scavenger of free radicals (Ames B N et al, 1981). Uric acid or its monoanion urate in its antioxidant function in blood may protect ascorbate from oxidation by chelation of iron (the formation of stable co-ordination complexes with iron ions) (Davies K J A et al., 1986). Uric acid is the end-product of purine metabolism in man and primates. It is believed to be responsible in part for the longevity of humans among primates and mammals because of its antioxidant properties (Ames B N et al., 1981; Cutler R G, 1984), perhaps concomitant with the antioxidant properties of ascorbic acid (Proctor P, 1970). High uric acid levels may have deleterious effects in humans besides being pathogenic in gout. It has been suspected that it may cause vascular disease and salt-sensitive hypertension in humans (Watanabe S et al., 2002). However, uric acid has revealed neuroprotective effects after experimental cerebral ischemia and uric acid has been administered intravenously in healthy humans without untoward effects to show an increase in serum free-radical scavenging capacity (Chamorro A et al., 2004). It has been shown in the art that urate anion free radical is produced in the one-electron oxidation of uric acid as an antioxidant and that ascorbic acid is able to reduce essentially all of the urate free radical produced; that is, the urate anion free radical can be scavenged by ascorbic acid (Maples K R et al., 1988; Simic M G et al. 1989). Uric acid may act critically in the repair of oxidative damage to DNA (Simic M G et al., 1989). Viewed edge-on, purines are essentially planar molecules which make them stack closely in the interior of double-stranded helices of DNA or DNA-RNA-hybrids (Rodwell V W, 1996). It is known that uric acid and xanthine (2,6-oxypurine) are difficulty soluble in water, while hypoxanthine is more soluble in water. Uric acid given orally is ineffective in elevating human serum uric acid levels (Spitsin S et al., 2001; Koprowisk H et al., 2001). Mononucleotides and hypoxanthine, but not xanthine, given orally was shown years ago to effectively raise serum uric acid levels both in normal adults and in gouty individuals (Clifford A J et al., 1976). It appears that both L-ascorbic acid and hypoxanthine are transported into cells by sodium ion-dependent active transport processes (Tsukaguchi H et al., 1999; Henler S S H, Rovik D, 2001; Griffith D A et al., 1993). Xanthine oxidase exists in the intestinal mucosa and liver to convert absorbed hypoxanthine to xanthine and uric acid before the resulting urate reaches the systemic circulation (Rodwell V W, 1996, p. 380). Absorbed hypoxanthine is quantitatively metabolized to end-products, primarily uric acid (Salati L M et al, 1984).

In AD patients and in healthy subjects, plasma and cerebrospinal fluid concentrations of ascorbic acid correlate highly and the levels also correlate with daily doses of ascorbic acid (Quinn J et al. 2003). Low cerebrospinal fluid levels of ascorbic acid correlate with low serum concentrations of ascorbic acid even in healthy subjects and therefore high serum levels have been advocated to ensure high cerebrospinal fluid concentrations (Tallaksen C M E et el., 1992). Of significance perhaps to the pathogenesis of AD, patients with AD have abnormally increased renal fractional excretion of uric acid associated with hypouricemia (Maesaka J K et al., 1993). Plasma antioxidants are similarly depleted in AD and in mild cognitive impairment (Rinaldi P et al. 2003). In 10 patients with AD-type dementia, mean urate values were low both in serum and in cerebrospinal fluid (Tohgi H et al., 1993). Cerebrospinal fluid levels of urate in man are generally only about 6 to 15% of the concomitant plasma or serum level of about 3 to 5 mg/dL, but higher in patients with gout with average plasma level of 7.36 mg/dL (Wolfson W Q et al., 1947).

Ascorbic acid intakes greater than 95 mg/day have been reported to have borderline significance of association with AD (Engelhart M J et al., 2002). However, study has not yet been reported to determine if supplemental combined intake of ascorbic acid or its sodium salt jointly with uric acid or an oxopurine precursor of uric acid like hypoxanthine may be effective in preventing or mitigating the Fenton-type oxidative damage from reaction of bivalent iron with hydrogen peroxide at low concentration in AD, in late onset MCI, or even in Down syndrome. In Down syndrome, subjects develop the neuropathologic cardinal features of AD by the age of 15-40 years (Burger P C et al., 1973; Sinet P M, 1982; Wisniewski K E et al., 1985).

In this trisomy 21 genetic disorder, superoxide dismutase activity is increased and the accumulation of cell hydrogen peroxide and cell oxidative damage by hydroxyl free radical formation catalyzed by reduced iron or copper likely leads to accelerated aging (Sinet P M, 1982; Kedziora J et al., 1988; Antila et al., 1989). Supplementary vitamins including ascorbic acid and minerals including iron and copper salts in megadoses have been used unsuccessfully in trials in school-aged children with Down syndrome (Bennett F C et al., 1983: Smith G F et al., 1984). Precursor of uric acid was not used in these trials.

Plasma mean levels of uric acid, hypoxanthine, and xanthine are elevated modestly in subjects with Down syndrome, with a mean urate level of 4.0 mg/dL compared with a normal mean urate level of 3.1 mg/dL in control individuals (Appleton M D et al., 1970). Similar modest serum uric acid elevations have been reported by other investigators (Fuller R W et al., 1962; Chapman M J et al., 1964). The increased urate levels are considered due to increased nucleoprotein breakdown and decreased renal excretion because of decreased renal function (Chapman M J et al., 1964; Coburn S P et al., 1967).

Amyotrophic lateral sclerosis is a degenerative disorder of motor neurons in the cortex, brainstem, and spinal cord. At least in some cases, mutations in superoxide dismutase genes may be responsible for the metalloenzyme having more dismutase activity and producing more hydrogen peroxide and oxygen and free radical toxicity (Rosen D R et al., 1993). Antioxidants including ascorbic acid have been tried as possible treatment with insufficient evidence of efficacy (Orrell R W et al., 2007). Trial of ascorbic acid or sodium ascorbate jointly with a purine precursor of uric acid or uric acid has not been reported apparently in amyotrophic lateral sclerosis. Wide fluctuations of ascorbic acid plasma levels with lesser fluctuations in cerebrospinal fluid levels occur in healthy subjects, amyotrophic lateral sclerosis subjects, and in Alzheimer's disease subjects (Paraskevas G P et al., 1997).

Oxidative stress with neuronal damage contributes to dopaminergic neuron degeneration in Parkinson's disease, a disease with characteristic degeneration of the pigmented cells in the substantia nigra and other brain stem dopaminergic cell groups. Dopamine action in the basal ganglia area is interfered with, reduced, or lost. Higher plasma urate concentrations may reduce the risk of Parkinson's disease according to a large cohort epidemiologic study in which the mean urate concentration was 5.7 mg/dL among cases and significantly higher at 6.1 mg/dL among control males (Weisskopf M G et al., 2007). The authors of this study suggested that high plasma urate levels may decrease the risk of Parkinson's disease in uric acid being neuroprotective. They raised the possibility that interventions to increase plasma urate may reduce the risk and delay the progression of Parkinson's disease. This disease most often is evident clinically after 50 years of age.

Catalase is normally absent or extremely low in human plasma (Halliwell B., 1989) and the “low” mean plasma level of hydrogen peroxide in adults may be as high as 34 or 38 micromolar (Varma S D et al., 1993; Smielecki J et al., 1996). Also, “free” iron in samples of cerebrospinal fluid from neurological patients have been reported to range from 3.5 to 24 micromolar, with a mean value of 9.4 micromolar (Gutteridge J M C et al., 1981).

### SUMMARY OF THE INVENTION

I postulate that low micromolar concentrations of hydrogen peroxide in cells and extracellular fluids may become iron-catalyzed to produce very toxic hydroxyl- or ferryl-free radicals that are site-specific and seminal in causing oxidative damage even early in Alzheimer's disease, amnestic mild cognitive impairment, Down syndrome, amyotrophic lateral sclerosis, and Parkinson's disease. A method to mitigate or prevent such oxidative changes in organic molecules by Fenton-type chemical reaction at low micromolar concentrations of hydrogen peroxide and bivalent iron is illustrated in saline solution, by antioxidant action of uric acid and ascorbic acid at physiologic pH. For this purpose, a novel and useful pharmacologic composition is provided to increase the antioxidant levels of urate and ascorbate in human extracellular fluids, by means of the absorption of hypoxanthine by active sodium-dependent intestinal transport as precursor to uric acid jointly with ascorbic acid. Potentially deleterious effects of formed urate anion free radicals in the antioxidant action of uric acid will be concomitantly mitigated by the administered ascorbate in this invention.

### DETAILED DESCRIPTION OF THE INVENTION

The chemicals hypoxanthine and L-ascorbic acid or the sodium salt of L-ascorbic acid in the pharmacologic composition in this invention are available from various commercial listed sources such as those listed in the directory: Chem Sources in U.S.A., 1999 edition, Clemson, S. C. 29633-1824, U.S.A. Sodium ascorbate USP as well as L-ascorbic acid USP in granular or powder form and hypoxanthine (6-oxypurine) are available from Spectrum Chemicals and Laboratory Products, Inc, Gardena, Calif. 90248-9985, for example.

All chemicals used in the following first 4 EXAMPLES for this invention were of analytical quality. A colorimeter was used to detect peroxidations of o-dianisidine, 9 mg/dL, as chromogenic hydrogen donor when oxidized, with its absorbance measured at 460 nm in saline solutions (140 mM NaCl, buffered by 3 mM phosphate salts at pH 7.4). Ferrous sulfate or human hemoglobin was added to control and to test solutions containing uric acid or ascorbic acid shortly before the addition of hydrogen peroxide, with oxidized molecules of dianisidine measured promptly. The saline solutions were mimetic of cerebrospinal fluid except devoid of possible confounding other molecules.

### EXAMPLE 1

Uric Acid Inhibition of Substrate Oxidations Induced by Reaction of Ferrous Sulfate (9.8 Micromolar) with 20 Micromolar Hydrogen Peroxide

| | Time after H₂O₂ | Absorbance Increase | % Inhibition |
|---|---|---|---|
| Control | 5 min. | 0.049 ± 0.004 | |
| | 10 min. | 0.049 ± 0.004 | |
| Urate, 6.0 mg/dL | 5 min. | 0.012 ± 0.003 | 75.3 ± 4.8** |
| | 10 min. | 0.014 ± 0.003 | 70.9 ± 4.5** |
| Urate, 0.5 mg/dL | 5 min. | 0.030 ± 0.003 | 36.8 ± 5.3** |
| | 10 min. | 0.034 ± 0.017 | 26.5 ± 5.5* |

Values are means ± s.e.m., n is 5.
Significance from control: P = *<0.01; **<0.001.

Comment: All incubations were done at 25-28° Celsius.

### EXAMPLE 2

Uric Acid Inhibition of Substrate Oxidation by Human Hemoglobin Level of 12 mg/dl in Reaction with 20 Micromolar Hydrogen Peroxide

| | Time | | |
|---|---|---|---|
| | after H₂O₂ | Absorbance Increase | % Inhibition |
| Control | 5 min. | 0.059 ± 0.002 | |
| | 10 min. | 0.084 ± 0.017 | |
| Urate, 6.0 mg/dL | 5 min. | 0.006 ± 0.002 | 90.6 ± 0.90*** |
| | 10 min. | 0.016 ± 0.005 | 80.3 ± 3.54** |
| Urate, 0.5 mg/dL | 5 min. | 0.028 ± 0.004 | 50.7 ± 7.56* |
| | 10 min. | 0.052 ± 0.009 | 38.8 ± 3.25** |

Values are means ± s.e.m., n is 4.
P = *<0.01, **<0.005, ***<0.001.

### EXAMPLE 3

Ascorbic Acid Inhibition at 1.6 mg/dl of Substrate Oxidation Induced by Reaction of Ferrous Sulfate (9.8 Micromolar) with 20 Micromolar Hydrogen Peroxide

| | Time | | |
|---|---|---|---|
| | after H₂O₂ | Absorbance Increase | % Inhibition |
| Control | 5 min. | 0.039 ± 0.007 | |
| | 10 min. | 0.039 ± 0.007 | |
| Ascorbate, 1.6 mg/dL | 5 min. | 0.000 ± 0.000 | 100.0 ± 0.0* |
| | 10 min. | 0.000 ± 0.000 | 100.0 ± 0.0* |

Values are means ± s.e.m., n = 4.
P = *<0.0001.

### EXAMPLE 4

Ascorbic Acid Inhibition at 1.6 mg/dl of Substrate Oxidation Induced by Human Hemoglobin Level of 12 mg//dl in Reaction with 20 Micromolar Hydrogen Peroxide

| | Time | | |
|---|---|---|---|
| | after H₂O₂ | Absorbance Increase | % Inhibition |
| Control | 5 min. | 0.050 ± 0.028 | |
| | 10 min. | 0.094 ± 0.006 | |
| Ascorbate, 1.6 mg/dL | 5 min. | 0.000 ± 0.000 | 100.0 ± 0.0* |
| | 10 min. | 0.001 ± 0.008 | 99.8 ± 1.2* |

Values are means ± s.e.m., n = 4.
P = *<0.0001.

Comments: The inhibition remained virtually complete in EXAMPLE 4 at 10 minutes of continued exposure to the hydrogen peroxide, in spite of greater absorbance increase or oxidation in the control solutions from continuing reaction of the peroxide with the iron that resided in the hemoglobin. The demonstrated hemoglobin-hydrogen peroxide reactions with absorbance increases confirm the Fenton-chemistry oxidation finding of Gutteridge (Gutteridge J M C., 1986). He used higher concentrations of hemoglobin and hydrogen peroxide (e.g. 0.67 or 0.8 mM of hydrogen peroxide) in phosphate-saline buffer with 2 hour incubations at 37° C.

A preferred embodiment of this invention employs a method which in millimole units contains 2.5-times more sodium ascorbate than hypoxanthine in capsule or tablet form. Their respective molecular weights are 198.1 and 136.1. Thus a preferred compositional mole ratio is supplied readily by blending the following amounts of sodium ascorbate USP and hypoxanthine as used in EXAMPLE 5. About 0.9 gram of hypoxanthine given orally was required to raise serum or plasma uric acid level by about 2.4 mg/dL in persons of near 70 kg in weight (0.1 mmole/kg: Clifford A J et al., 1976). A more tolerable and non-acidity effect in the digestive tract is accomplished by use of sodium ascorbate in place of ascorbic acid (Hendler S S et al., 2001). Alternatively, when L-ascorbic acid (of molecular weight of 176.1) is employed in the same compositional mole ratio of 2.5 to 1, a slightly lesser weight amount of ascorbic acid is used in a preferred embodiment.

### EXAMPLE 5

For avg. No. 00-sized gelatin capsules:

Sodium ascorbate granules USP=470.5 mg (2.38 mmoles)
Hypoxanthine powder=129.3 mg (0.95 mmole)
Approximate net weight=600 mg per capsule

For example, mix 4.70 g of sodium ascorbate granules and 1.29 g of hypoxanthine by blending for about a minute. Then, fill No. 00-sized gelatin capsules to an average net weight of approximately 600 mg per blend per capsule, to make 9 plus filled capsules. Two or three capsules per serving may be taken by month with liquid usually once or twice daily to raise plasma urate and ascorbate levels conjointly.

While the invention has been described with reference to a specific embodiment, it will be appreciated that numerous variations, modifications, and embodiments are possible, and accordingly, all such variations, modifications, and embodiments are to be regarded as being within the spirit and scope of the invention.

### PUBLICATIONS

Ames B H et al. Uric acid provides an antioxidant defense in humans against oxidant- and radical-caused aging and cancer: A hypothesis. Proc. Natl. Acad. Sci. USA 1981; 78: 6858-6862
Antila E et al. On the etiopathogenesis and therapy of Down syndrome. Int. J. Dev. Biol. 1989; 33: 181-188.
Appleton M D et al. Plasma urate levels in mongolism. Am. J. Ment. Defic. 1970; 74: 196-199.
Bennett F C et al. Vitamin and mineral supplementation in Down's syndrome. Pediatrics. 1983; 72: 707-713.
Brookmeyer R et al. Projections of Alzheimer's disease in the United States and the public health impact of delaying disease onset. Am. J. Public Health. 1998; 88:1337-1342.
Burger P C et al. The development of the pathologic changes of Alzheimer's disease and senile dementia in patients with Down's syndrome. Am. J. Path. 1973; 73: 457-476.
Chapman M J. Uric acid in Down's disease. J. Ment. Defic. Res. 1964; 8: 119-124.
Christen Y. Oxidative stress and Alzheimer's disease. Am. J. Clin. Nutr. 2000; 71(suppl): 621S-629S.
Clifford A J et al Effect of oral purines on serum and urinary uric acid of normal, hyperuricemic and gouty humans. J. Nutr. 1976; 106: 428-450.
Coburn S P et al. Clearance of uric acid, urea, and creatinine in Down's syndrome. J. Applied Physiol. 1967; 23: 579-580.
Connor J R et al. A quantitative analysis of isoferritins in select regions of aged, Parkinsonian, and Alzheimer's diseased brains. J. Neurochem. 1995; 65:717-724.
Cutler R G. Urate and ascorbate: their possible roles as antioxidant in determining longevity of mammalian species. Arch. Gerontol. Geriatr. 1984; 3: 321-348.
Davies K J A et al. Uric acid-iron ion complexes A new aspect of the antioxidant functions of uric acid. Biochem. J. 1986; 235: 747-754.
Engelhart M J et al. Dietary intake of antioxidants and risk of Alzheimer's disease. JAMA. 2002; 287: 3223-3229.
Evans D A et al. Prevalence of Alzheimer's disease in a community population of older persons Higher than previously reported. JAMA. 1989; 262: 2551-2556.
Fuller R W et al. Serum uric acid in mongolism. Science. 1962; 137: 368-369.
Goedert M et al. A century of Alzheimer's disease. Science. 2006; 314: 777-781.
Goodman 1. Alzheimer's disease A clinico-pathologic analysis of twenty-three cases with a theory on pathogenesis. J. Nervous Mental Disease. 1953; 117: 97-130.
Griffith D A et al. High affinity sodium-dependent nucleobase transport in cultured renal epithelial cells (LLC-PK₁). J. Biol. Chem. 1993; 268: 20085-20090.
Gutteridge J M C. Iron promoters of the Fenton reaction and lipid peroxidation can be released from haemoglobin by peroxides. FEBS Lett. 1986; 201: 291-295.
Halliwell B. Oxidants and the central nervous system: some fundamental questions Is oxidant damage relevant to Parkinson's disease, Alzheimer's disease, traumatic injury or stroke? ActaNeurol. Scand. 1989; 126: 23-33.
Hendler S S et al.(chief eds): PDR for Nutritional Supplements. Vitamin C. Medical Economics, Montvale, N.J., 2001: pp 486-498.
Hershey C O et al. Cerebrospinal fluid trace element content in dementia: Clinical, radiologic, and pathologic correlations. Neurology. 1983; 33: 13501353.
Imlay J A. Toxic DNA damage by hydrogen peroxide through the Fenton reaction in vivo and in vitro. Science. 1988; 240: 640-642.
Kedziora J. et al. Down's syndrome: a pathology involving the lack of balance of reactive oxygen species. Free Radical Biol. Med. 1988; 4: 317-330.
Koprowski H et al. Prospects for the treatment of multiple sclerosis by raising serum levels of uric acid, a scavenger of peroxynitrite. Ann. Neurol. 2001; 49: 139.
Maesaka J K et al. Hypouricemia, abnormal renal tubular urate transport, and plasma natriuretic factor(s) in patients with Alzheimer's disease. J. Am. Geriatr. Soc. 1993; 41: 501-506.
Maples K R. Free radical metabolite of uric acid. J. Biol. Chem 1988; 263:1709-1712.
Markesbery W R. Damage to lipids, proteins, DNA, and RNA in mild cognitive impairment. Arch. Neurol. 2007; 64: 954-956.
Merocci P et al. Lymphocyte oxidative DNA damage and plasma antioxidants in. Alzheimer disease. Arch. Neurol. 2002; 59: 794-798.
Nunomura A et al. Involvement of oxidative stress in Alzheimer's disease. J. Neuropathol. Exp. Neurol. 2006; 65: 631-640.
Orrell R W et al. Antioxidant treatment for amyotrophic lateral sclerosis/motor neuron disease. Cochrane Database of Systematic Rev. 2007; 1: Art. No. CD 002829. DO1.
Paraskevas G P et al. Ascorbate in healthy subjects, amyotrophic lateral sclerosis and Alzheimer's disease. Acta Neurol. Scand. 1997; 96: 88-90.
Proctor P. Similar functions of uric acid and ascorbate in man? Nature. 1970; 228:868.
Quinn J. et al. Antioxidants in Alzheimer's disease-vitamin C delivery to a demanding brain. J. Alzheimer's disease. 2003; 5: 309-313.
Rinaldi P. et al. Plasma antioxidants are similarly depleted in mild cognitive impairment and in Alzheimer's disease. Neurobiol. Aging. 2003; 24: 915-919.
Roberson E D et al. 100 years and-counting: prospects for defeating Alzheimer's disease. Science. 2006; 314: 781-784.
Rodwell V W. Nucleotides. Metabolism of purine and pyrimidine nucleotides.: in Murray R K et al. (eds): Harper's Biochemistry 24th ed, Stamford, Appleton & Lange, 1996, p 360 and p 380.
Rosen D R et al. Mutations in Cu/Zn superoxide dismutase gene are associated with amyotrophic lateral sclerosis. Nature. 1993; 362: 59-62.
Salati L M et al. Absorption and metabolism of adenosine, adenosine-5′-monophospate, adenosine and hypoxanthine by the isolated vascularly perfused rat small intestine. J. Nutri. 1984; 114: 753-760.
Smielecki J. et al. The influence of electrical cardioversion on'superoxide anions (O₂⁻) production by polymorphonuclear neutrophils, hydrogen peroxide (H₂O₂) plasma level and malondialdehyde serum concentration. Int. J. Cardiology. 1996; 56: 137-143.
Simic M G et al. Antioxidation mechanisms of uric acid. J. Am. Chem. Soc. 1989; 111: 5778-5782.
Sinet P M. Metabolism of oxygen derivatives in Down's syndrome. Ann. New York Acad. Sci. 1982; 396: 83-94.
Smith G F et al. Use of megadoses of vitamins with minerals in Down syndrome. J. Pediatr. 1984; 105: 228-234.
Smith M A et al. Iron accumulation in Alzheimer disease is a source of redox-generated free radicals. Proc. Natl. Acad. Sci. 1997; 94: 9866-9868.
Spitsin S et al. Inactivation of peroxynitrite in multiple sclerosis patients after oral administration of inosine may suggest possible approaches to therapy of the disease. Multiple Sclerosis. 2001; 7: 313-319.
Tallaksen C M E et al. Concentrations of the water-soluble vitamins thiamin, ascorbic acid, and folic acid in serum and cerebrospinal fluid of healthy individuals. Am. J. Clin. Nutr. 1992; 56: 559-564.
Todorich B M et al. Redox metals in Alzheimer's disease. Ann. New York Acad. Sci. 2004; 1012: 171-178.
Tohgi H et al. The urate and xanthine concentrations in the cerebrospinal fluid in patients with vascular dementia of the Binswanger type, Alzheimer type dementia, and Parkinson's disease. J. Neural. Transm. [P-D Sect]. 1993; 6: 119-126.
Tsukaguchi H et al. A family of mammalian Na⁺-dependent 1-ascorbic acid transporters. Nature. 1999; 399: 70-76.
Watanabe S et al. Uric acid, hominoid evolution, and the pathogenesis of salt-sensitivity. Hypertension. 2002; 40: 355-360.
Waugh W H. Simplified method to assay total plasma peroxidase activity and ferriheme products in sickle cell anemia, with initial results in assessing clinical severity in a trial with citrulline therapy. J. Pediatr. Hematol. Oncol. 2003; 25: 831-834.
Waugh W H. Cognitive declines therapy by iron burden reduction. Arch. Intern. Med. 2007; 167: 1098.
Weisskopf M G et al. Plasma urate and risk of Parkinson's disease. Am. J. Epidemiology. 2007; 166: 561-567.
Wisniewski K E et al. Alzheimer's disease in Down's syndrome: Clinicopathologic studies. Neurology. 1985; 35: 957-961.
Wolfson W Q et al. The transport and excretion of uric acid in man. I. True uric acid in normal cerebrospinal fluid, in plasma, and in ultrafiltrates of plasma. J. Clin. Invest. 1947;26: 991-994.
Varma S D et al. Hydrogen peroxide in human blood. Free Rad. Res. Comms. 1991; 14: 125-131.

1. A method of improving the health of a subject to increase the extracellular levels of urate and ascorbate conjointly for improved antioxidant activity by administering the pharmacologic combined composition of hypoxanthine and ascorbate.

2. A method of preventing disease and mitigating early disease damage in a subject to increase the extracellular levels of urate and ascorbate conjointly for improved antioxidant activity comprising administering the pharmacologic combined composition of hypoxanthine and ascorbate.

3. The method according to claim 2, wherein the subject has a serum usual fasting uric acid level below 8.0 mg/deciliter.

4. The method according to claim 2, wherein the subject has signs of Alzheimer's disease.

5. The method according to claim 2, wherein the subject has signs of amnestic mild cognitive impairment.

6. The method according to claim 2, wherein the subject has signs of Down syndrome.

7. The method according to claim 2, wherein the subject has signs of amyotrophic lateral sclerosis.

8. The method according to claim 2, wherein the subject has signs of Parkinson's disease.

9. The method according to claim 1, wherein the subject has a risk factor for progressive degenerative disease of the central nervous system.

10. The method according to claim 1, wherein the combined pharmacologic composition includes hypoxanthine conjointly with sodium L-ascorbate.

11. The method according to claim 1, wherein the combined pharmacologic composition includes hypoxanthine conjointly with L-ascorbic acid.

12. The method according to claim 1, wherein the combined pharmacologic composition is administered orally usually at least once daily.

13. The method according to claim 1, wherein the combined pharmacologic composition is administered in a form selected from the group consisting of capsule, tablet, and liquid.

14. The method according to claim 2, wherein the subject has a risk factor for progressive degenerative disease of the central nervous system.

15. The method according to claim 2, wherein the combined pharmacologic composition includes hypoxanthine conjointly with sodium L-ascorbate.

16. The method according to claim 2, wherein the combined pharmacologic composition includes hypoxanthine conjointly with L-ascorbic acid.

17. The method according to claim 2, wherein the combined pharmacologic composition is administered orally at least once daily.

18. The method according to claim 2, wherein the combined pharmacologic composition is administered in a form selected from the group consisting of capsule, tablet, and liquid.
